(19) United States
(12) Patent Application Publication
(10) Pub. No.: US 2023/0215580 A1
(43) Pub. Date: Jul. 6, 2023

(54) INFORMATION PROCESSING METHOD, INFORMATION PROCESSING DEVICE, AND NON-TRANSITORY COMPUTER READABLE RECORDING MEDIUM

(71) Applicant: Panasonic Intellectual Property Corporation of America, Torrance, CA (US)

(72) Inventors: Kotaro SAKATA, Osaka (JP); Tetsuji FUCHIKAMI, Osaka (JP)

(21) Appl. No.: 18/119,448

(22) Filed: Mar. 9, 2023

Related U.S. Application Data
(63) Continuation of application No. PCT/JP2021/023986, filed on Jun. 24, 2021.

(30) Foreign Application Priority Data
Sep. 17, 2020 (JP) 2020-156044

(51) Int. Cl. G16H 50/30; G16H 20/00; G16B 25/00

(52) U.S. Cl. CPC G16H 50/30; G16B 25/00; G16H 20/00

(57) ABSTRACT

This information processing method specifies a disease that a user is likely to suffer from, based on gene analysis data of a user; analyzes behavior history data of the user and operation history data of an apparatus; generates first lifestyle pattern data indicating a lifestyle pattern of the user up to present; executes a simulation for causing a digital twin of the user and a digital twin of the apparatus to operate in cyberspace based on the first lifestyle pattern data, standard lifestyle pattern data, and the operation history data; generates second lifestyle pattern data in which a future lifestyle pattern of the user is predicted, from an execution result of the simulation; and calculates a future illness risk of the user for the specified disease based on the second lifestyle pattern data.

### TECHNICAL FIELD

The present disclosure relates to a technique for simulating a lifestyle pattern of a user using a digital twin.

### BACKGROUND ART

In recent years, a technique of performing various simulations using a digital twin that realistically expresses an existing object or person in a cyberspace has attracted attention. For example, Patent Literature 1 discloses generating a digital twin of a vehicle, executing one or a plurality of simulations based on the generated digital twin, and generating evaluation data describing a price of an insurance contract of the vehicle based on an execution result of the one or the plurality of simulations.

In recent years, with the development of gene analysis technique, there is known a technique for analyzing trait information such as a user’s diathesis from user’s genetic information and notifying the user of an analysis result. For example, Patent Literature 2 discloses a technique of referring to user’s diathesis information determined according to a genetic testing result of the user, generating an avatar image of the user in a form corresponding to the user’s diathesis information, and displaying the generated avatar image.

However, in the above-described conventional techniques, no future illness risk of the user is considered, and thus further improvement is required.

### CITATION LIST

### Patent Literature

Patent Literature 1: JP 2020-13557 A
Patent Literature 2: JP 2016-71721 A

### SUMMARY OF INVENTION

The present disclosure has been made to solve the above problem, and an object thereof is to provide a technique of predicting a future illness risk of a user.

An information processing method according to an aspect of the present disclosure includes, by a computer, generating, in a cyberspace, a digital twin of a user and a digital twin of an apparatus installed in a residence of the user, based on real-world data, acquiring behavior history data indicating a behavior history of the user and operation history data indicating an operation history of the apparatus, specifying a disease that the user is likely to suffer from, based on gene analysis data of the user, analyzing the behavior history data and the operation history data to generate first lifestyle pattern data indicating a lifestyle pattern of the user up to present, executing a simulation for causing the digital twin of the user and the digital twin of the apparatus to operate in the cyberspace based on the first lifestyle pattern data, standard lifestyle pattern data indicating a standard lifestyle pattern in accordance with a future life stage, and the operation history data, generating second lifestyle pattern data in which a future lifestyle pattern of the user is predicted from an execution result of the simulation, calculating a future illness risk of the user for the specified disease based on the second lifestyle pattern data, and outputting the illness risk.

According to the present disclosure, a future illness risk of a user can be predicted.

### BRIEF DESCRIPTION OF DRAWINGS

FIG. 1 is an overall configuration diagram of an information processing system according to an embodiment of the present disclosure.

FIG. 2 is a block diagram illustrating an example of a configuration of a server illustrated in FIG. 1.

FIG. 3 is a block diagram illustrating an example of a configuration of a sensor device.

FIG. 4 is a block diagram illustrating an example of a configuration of an apparatus.

FIG. 5 is a block diagram illustrating an example of a configuration of a terminal device.

FIG. 6 is a sequence diagram illustrating data transmission and reception in the sensor device, the apparatus, and the server.

FIG. 7 is a flowchart presenting an example of processing by the server illustrated in FIG. 1.

FIG. 8 is a view illustrating an example of a digital twin of a residence.

FIG. 9 is a view illustrating an example of a digital twin of a residence.

FIG. 10 is a view illustrating an example of a digital twin in a region.

FIG. 11 is an explanatory view of generation processing of first lifestyle pattern data.

FIG. 12 is a view illustrating an example of a data configuration of lifestyle pattern data of each day.

FIG. 13 is a view illustrating an example of a data configuration of a plurality of pieces of lifestyle pattern data.

FIG. 14 is a view illustrating an example of a life stage.

FIG. 15 is an explanatory view of simulation.

FIG. 16 is an explanatory view of processing of calculating a future illness risk.

FIG. 17 is a view illustrating a presentation screen.

FIG. 18 is a view illustrating a presentation screen according to another example.

FIG. 19 is a view illustrating a presentation screen according to still another example.

FIG. 20 is a view illustrating a presentation screen according to yet another example.

### DESCRIPTION OF EMBODIMENTS

### Knowledge Underlying Present Disclosure

In recent years, techniques for analyzing human genes have become high in speed and low in cost. With this, the user can easily have a genetic test at home or the like. Such genetic test enables determination as to whether or not the user has a diathesis to develop a specific disease such as a lifestyle disease.

However, just because the user has a diathesis to easily develop a specific disease does not mean the user necessarily develops the specific disease. For example, improvement of the lifestyle pattern in the future can reduce the illness risk for a specific disease. For this purpose, it is effective to predict a future lifestyle pattern of the user.

However, there has been no technique for predicting a future lifestyle pattern of a user and predicting a future illness risk of the user based on the predicted future lifestyle pattern and a gene analysis result.

For example, in Patent Literature 1 described above, a digital twin of a vehicle is merely generated, and a digital twin of a user is not generated. In Patent Literature 2 described above, an avatar image having a form corresponding to diathesis information of the user at the time of genetic test is merely generated, and a future illness risk is not predicted.

Therefore, the present inventor has obtained knowledge that a future lifestyle pattern of a user can be predicted by generating, in the cyberspace (computer space), a digital twin of the user and a digital twin of an apparatus or the like present in a residence of the user, and operating the generated digital twins in the cyberspace. Then, the present inventor has obtained knowledge that the future illness risk of the user for a disease can be predicted by using the predicted future lifestyle pattern and the user’s gene analysis result, and has conceived the aspects given below.

An information processing method according to an aspect of the present disclosure includes, by a computer, generating, in a cyberspace, a digital twin of a user and a digital twin of an apparatus installed in a residence of the user, based on real-world data, acquiring behavior history data indicating a behavior history of the user and operation history data indicating an operation history of the apparatus, specifying a disease that the user is likely to suffer from, based on gene analysis data of the user, analyzing the behavior history data and the operation history data to generate first lifestyle pattern data indicating a lifestyle pattern of the user up to present, executing a simulation for causing the digital twin of the user and the digital twin of the apparatus to operate in the cyberspace based on the first lifestyle pattern data, standard lifestyle pattern data indicating a standard lifestyle pattern in accordance with a future life stage, and the operation history data, generating second lifestyle pattern data in which a future lifestyle pattern of the user is predicted from an execution result of the simulation, calculating a future illness risk of the user for the specified disease based on the second lifestyle pattern data, and outputting the illness risk.

According to the present configuration, a disease that the user is likely to have is specified from the gene analysis data of the user. The first lifestyle pattern data indicating the lifestyle pattern of the user up to the present is generated from the behavior history data of the user and the operation history data of the apparatus. A simulation of causing the digital twin of the user and the digital twin of the apparatus to operate in the cyberspace based on the generated first lifestyle pattern data, the standard lifestyle pattern data in accordance with the future life stage, and the operation history data is executed. The second lifestyle pattern data for predicting the future lifestyle pattern of the user is generated from the simulation execution result. An illness risk of having the specified disease in the future is calculated based on the generated second lifestyle pattern data, and the calculated illness risk is output. Therefore, the present configuration can predict the illness risk of the disease that the user is likely to have in the future. By presenting the future illness risk to the user, it is possible to give the user an opportunity to review the current lifestyle pattern. This allows the user to reduce the future illness risk.

In the information processing method described above, a digital twin of the residence may be included in the cyberspace.

According to the present configuration, since the digital twin of the residence is included, behavior of the user in the residence can be simulated, and the prediction accuracy of the future lifestyle pattern of the user can be improved.

The information processing method described above may further include generating an improvement plan of the lifestyle pattern of the user based on the second lifestyle pattern data and the illness risk, in which in the outputting, the improvement plan may be further output.

According to the present configuration, since the improvement plan of the lifestyle pattern of the user is output, the lifestyle pattern for reducing the illness risk can be presented to the user.

In the information processing method described above, the improvement plan may include exercise information indicating exercise recommended for reducing the illness risk.

According to the present configuration, it is possible to present a preferable exercise for reducing the illness risk to the user.

In the information processing method described above, in calculating the illness risk, an illness risk within one or more future periods may be calculated.

According to the present configuration, it is possible to present the user with how much illness risk will exist at which time point in the future.

In the information processing method described above, the disease may be a lifestyle disease.

According to the present configuration, the illness risk for the lifestyle disease can be presented to the user.

In the information processing method described above, in generating the second lifestyle pattern data, a daily lifestyle pattern from the present to a predetermined future time point may be predicted.

According to the present configuration, since the future lifestyle pattern is predicted daily, the future lifestyle pattern can be finely predicted.

In the information processing method described above, the real-world data may include attribute data of the user and position data of the apparatus.

According to the present configuration, since the real-world data includes the attribute data of the user and the position data of the apparatus, it is possible to accurately generate the digital twin of the user and accurately arrange the digital twin of the apparatus.

In the information processing method described above, in executing the simulation, a simulation of causing the digital twin of the user to operate in the cyberspace based on the first lifestyle pattern data and the standard lifestyle pattern data, and causing the digital twin of the apparatus to operate in the cyberspace based on the operation history data may be executed.

According to the present configuration, since the simulation of causing the digital twin of the user to operate in the cyberspace based on the first lifestyle pattern data and the standard lifestyle pattern data, and causing the digital twin of the apparatus to operate in the cyberspace based on the operation history data of the apparatus is executed, the future lifestyle pattern of the user can be accurately predicted.

The present disclosure can be also implemented as a program for causing a computer to execute each characteristic configuration included in such an information processing method or as an information processing system operated by the program. It is needless to say that such a computer program can be distributed using a computer-readable non-transitory recording medium such as a CD-ROM, or via a communication network such as the Internet.

An embodiment described below illustrates a specific example of the present disclosure. Numerical values, shapes, constituent elements, steps, order of steps, and the like shown in the embodiment below are merely examples, and are not intended to limit the present disclosure. Furthermore, among constituent elements in the embodiment below, a constituent element that is not described in an independent claim indicating the highest concept is described as an optional constituent element. In all the embodiments, respective contents can be combined.

### (Embodiment)

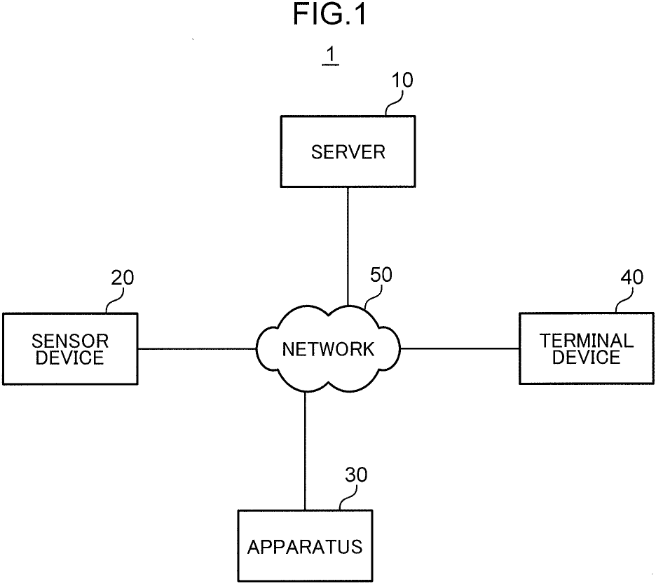

FIG. 1 is an overall configuration diagram of an information processing system 1 according to an embodiment of the present disclosure. The information processing system 1 includes a server 10, a sensor device 20, an apparatus 30, and a terminal device 40. The server 10, the sensor device 20, the apparatus 30, and the terminal device 40 are communicably connected to one another via a network 50. The network 50 is, for example, a wide-area communication network including the Internet and a mobile phone communication network. Furthermore, the network 50 may include a local area network.

The server 10 is a cloud server including one or more computers, for example. The server 10 receives sensing data from the sensor device 20. The server 10 receives operation data from the apparatus 30. The server 10 transmits, to the terminal device 40, presentation data including a future illness risk for a disease of a user having an illness risk and an improvement plan of a lifestyle pattern.

The sensor device 20 detects sensing data necessary for detecting behavior of the user. The sensor device 20 is a mobile terminal such as a smart watch, a smartphone, or a tablet terminal, for example. The sensing data includes, for example, position data of the user, biological data of the user, imaging data of the user, and measurement time. The sensor device 20 may be a camera installed in the residence. Furthermore, the sensor device 20 may be an odor sensor installed in the residence.

The apparatus 30 is an electric apparatus installed in the residence of the user. The electric apparatus is, for example, an air conditioning apparatus, a cooking apparatus such as an oven, or household electric apparatuses such as a refrigerator, a washing machine, a television set, a smart speaker, an audio apparatus, a DVD recorder, or a Blu-ray recorder. In a case where the power is in an on state, for example, the apparatus 30 transmits the operation data to the server 10 at a predetermined sampling cycle.

The terminal device 40 is a device that outputs the presentation data transmitted from the server 10. The terminal device 40 is, for example, a desktop computer installed in the residence of the user, a mobile terminal (smartphone or tablet terminal) carried by the user, or the like. The presentation data may be displayed on the apparatus 30 having a display. In a case where the terminal device 40 includes a mobile terminal, this mobile terminal may include the functions of the sensor device 20 and the terminal device 40.

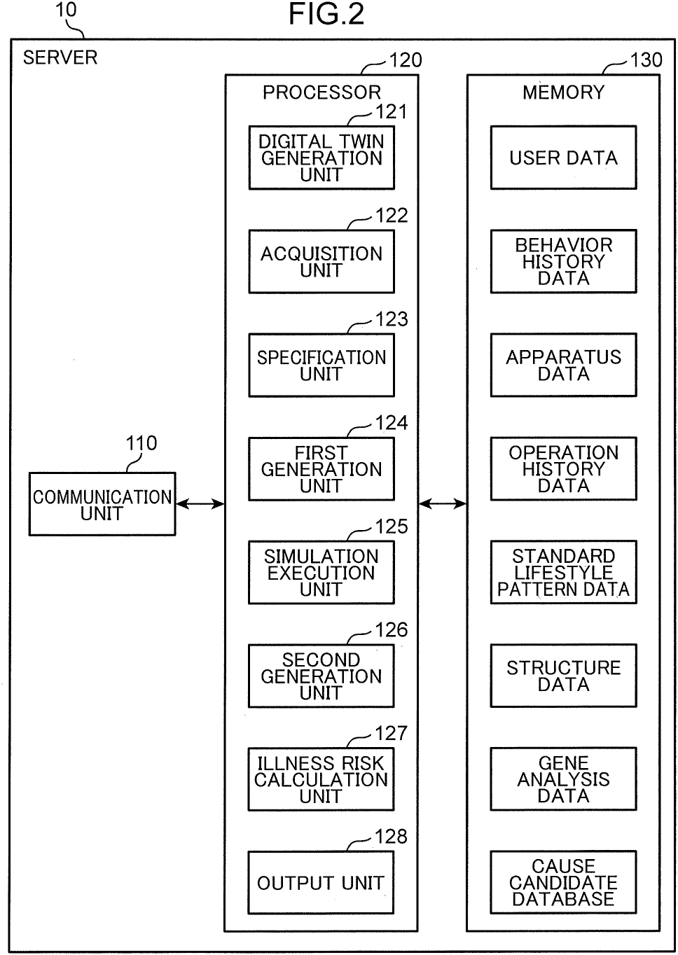

FIG. 2 is a block diagram illustrating an example of the configuration of the server 10 illustrated in FIG. 1. The server 10 includes a communication unit 110, a processor 120, and a memory 130. The communication unit 110 includes a communication circuit that connects the server 10 to the network 50. The communication unit 110 receives the sensing data from the sensor device 20, receives the operation data from the apparatus 30, and transmits the presentation data to the terminal device 40.

The processor 120 includes a processor such as a CPU. The processor 120 includes a digital twin generation unit 121, an acquisition unit 122, a specification unit 123, a first generation unit 124, a simulation execution unit 125, a second generation unit 126, an illness risk calculation unit 127, and an output unit 128. Each block included in the processor 120 may be implemented by the CPU executing a predetermined program, or may be configured by a dedicated hardware circuit.

The digital twin generation unit 121 generates a digital twin of the user in the cyberspace by using user data. The user data includes attribute data of the user such as age, gender, height, and weight, for example. This attribute data is basic data necessary for generating the digital twin of the user.

The digital twin generation unit 121 generates a digital twin of the apparatus 30 in the cyberspace by using apparatus data. The apparatus data includes, for example, the type of the apparatus 30, data indicating an installation position of the apparatus 30 in the residence, data indicating the model of the apparatus 30, and a function indicating an input/output relationship between an operation input to the apparatus and an output for the operation.

The digital twin generation unit 121 generates a digital twin of the residence in the cyberspace by using structure data three-dimensionally indicating the structure of the residence. The structure data is, for example, computer-aided-design (CAD) data and building information modeling (BIM) data. The structure data of the residence is data for reproducing a solid model of the real residence in the cyberspace. The structure data of the residence includes structure data of the appearance, the floor plan, and the garden of the residence. The digital twin generation unit 121 may generate a digital twin of a certain region including the residence of the user. In this case, the digital twin generation unit 121 is only required to generate a digital twin of this region by using the structure data of this region.

Note that, since Dymola, MapleSim, Simulink, and the like are known as software for generating a digital twin, the digital twin generation unit 121 may generate a digital twin by using these pieces of software.

When the communication unit 110 receives the sensing data transmitted from the sensor device 20, the acquisition unit 122 acquires the sensing data from the communication unit 110 and stores the acquired sensing data into the memory 130 as behavior history data. The behavior history data is data in which, for example, a sensor value included in the sensing data, the type of the sensor device 20 that has transmitted the sensing data, and a time stamp are associated with one another. The sensor value includes, for example, position data of the user and biological data of the user.

When the communication unit 110 receives the operation data transmitted from the apparatus 30, the acquisition unit 122 acquires the operation data from the communication unit 110 and stores the acquired operation data into the memory 130 as operation history data. The operation history data is data in which, for example, an operation value indicated by the operation data, the type of the apparatus 30 that has transmitted the operation data, and a time stamp are associated with one another. The operation value is, for example, power-on, power-off, setting content, and the like. For example, in the case of the air conditioning apparatus, the setting content includes a setting temperature and an operation mode such as cooling and heating.

When the simulation execution unit 125 executes the simulation, the acquisition unit 122 acquires the behavior history data and the operation history data from the memory 130.

The specification unit 123 acquires gene analysis data from the memory 130, and specifies a disease with which the user has an illness risk based on the acquired gene analysis data. The gene analysis data includes a disease-associated SNP that is single nucleotide polymorphism (SNP) associated with a specific disease and the type of the disease-associated SNP.

Human base sequences are identical by 99.9%, but different by 0.1%. This difference causes a difference in appearance, ability, diathesis, and the like. When a difference in base sequence appears at a frequency of 1% or more in a certain human population, the difference in base sequence is called polymorphism. There are various types of polymorphism, and among them, SNP is one in which one base is replaced by another base. Although there are many of SNP, it has been indicated that a specific SNP is associated with a specific disease. Such SNP is called a disease-associated SNP.

The type of SNP is a combination of, for example, the SNP inherited from the father such as AA, AG, and GG and the SNP inherited from the mother. From this type of SNP, it is possible to specify the risk in which the user will have a certain disease in the future.

Therefore, the specification unit 123 specifies a disease that the user will possibly have, from the disease-associated SNP and the type of the disease-associated SNP. Furthermore, the specification unit 123 specifies the disease that the user will possibly have and specifies an illness risk. The illness risk indicates the probability of having a specific disease, and is expressed by, for example, a numerical value of 0 to 100. The specific disease is, for example, a lifestyle disease. Examples of the lifestyle disease include arteriosclerosis, hypertension, diabetes, osteoporosis, and dementia.

The server 10 is only required to acquire gene analysis data of the user in advance and store the data in the memory 130. The gene analysis data may be generated based on a test result by an external institution, for example, or may be measured at the user’s home. As a method for measuring SNP and the SNP type, it is possible to adopt, for example, restriction fragment length polymorphism (RFLP), single strand conformation polymorphism (SSCP), SSCP, TaqManPCR, SNaP Shot, Invader, a mass spectrometry method, and a method using a DNA microarray.

The first generation unit 124 analyzes the behavior history data and the operation history data acquired by the acquisition unit 122 from the memory 130, and generates first lifestyle pattern data indicating a lifestyle pattern of the user up to the present. In the present embodiment, the first generation unit 124 generates lifestyle pattern data of each day within a period in which the behavior history data is acquired. For example, assuming that the acquisition period of the behavior history data is 5 years, lifestyle pattern data for 365 days × 5 years is generated. Then, the first generation unit 124 is only required to generate the first lifestyle pattern data by bringing together the lifestyle pattern data of each day, for example, by day of the week.

The simulation execution unit 125 executes a simulation of causing the digital twin of the user to operate in the cyberspace by using the first lifestyle pattern data and the standard lifestyle pattern data indicating a standard lifestyle pattern in accordance with the future life stage, and causing the digital twin of the apparatus 30 to operate in the cyberspace based on the operation history data.

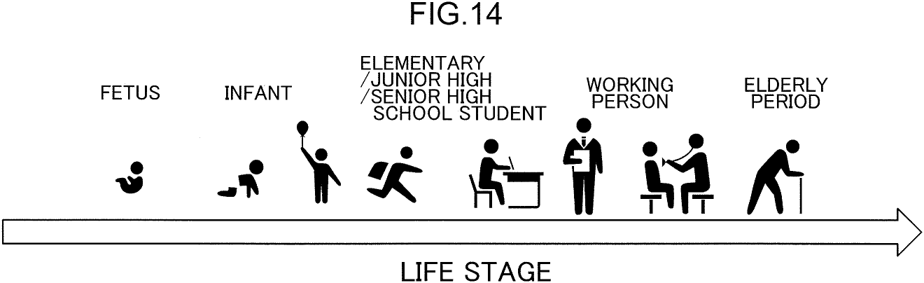

The standard lifestyle pattern data is data indicating a lifestyle pattern of general people for each age. FIG. 14 is a view illustrating an example of a life stage. The life stage refers to a stage of life that changes with age. For example, the life stage includes stages such as a fetus, an infant, an elementary school student, a junior high school student, a senior high school student, a working person, and an elderly period.

A Japanese person has a life stage including graduating from a university at the age of 22, working as a working person from the age of 23, and retiring at the age of 65. With age, a human has a shorter sleep hours, a smaller amount of meal, and less burned calories for behavior with a decrease in basal metabolic rate.

Therefore, the standard lifestyle pattern data includes lifestyle pattern data of general people created for each age in consideration of such a life stage. The standard lifestyle pattern data may include, for example, age-appropriate lifestyle pattern data for each day of the week. Furthermore, the standard lifestyle pattern data may include an age-appropriate basal metabolic rate and standard consumed calories per day.

Since the first lifestyle pattern data described above indicates the lifestyle pattern of the user up to the present, in order to predict the future lifestyle pattern, it is necessary to cause the digital twin of the user to operate in the cyberspace in accordance with the expected future lifestyle pattern. Therefore, the simulation execution unit 125 uses the standard lifestyle pattern data when executing the simulation.

The simulation execution unit 125 executes a simulation in a simulation period, for example, from the present (simulation execution time) to a certain future time point (for example, 5 years later). For example, the simulation execution unit 125 is only required to execute the simulation in units of one day during the simulation period. For example, when executing a simulation of a certain day in two years, the simulation execution unit 125 corrects the first lifestyle pattern data of the day of the week of the corresponding day by using the standard lifestyle pattern data of the corresponding day of the week in two years. Then, the simulation execution unit 125 is only required to cause the digital twin of the user to operate in the cyberspace using the corrected first lifestyle pattern data.

Furthermore, by using the operation history data brought together for each day of the week, the simulation execution unit 125 causes the digital twin of the apparatus 30 to operate. For example, in a case of executing a simulation on a certain day in two years later, the simulation execution unit 125 is only required to cause the digital twin of the apparatus 30 to operate in the cyberspace using the operation history data corresponding to the day of the week of the day.

The second generation unit 126 generates second lifestyle pattern data in which the future lifestyle pattern of the user is predicted from the simulation execution result. The second lifestyle pattern data includes, for example, lifestyle pattern data in which the behavior of the digital twin of the user for each day of the simulation period is listed in time series. The lifestyle pattern data of each day is data in which the user’s behavior of each day such as sleeping from 0:00 to 6:00 and eating from 5:30 to 7:00 is arranged in time series. Here, similarly to the first generation unit 124, the second generation unit 126 is only required to specify the user’s behavior using the behavior history data and the operation history data included in the simulation execution result, and describe the specified behavior in time series, thereby generating the lifestyle pattern data for each day of the simulation period.

The illness risk calculation unit 127 calculates a future illness risk of the user for the disease specified by the specification unit 123 based on the second lifestyle pattern data. For example, the illness risk calculation unit 127 specifies one or more cause candidates of the disease specified by the specification unit 123 by referring to a cause candidate database in which a plurality of diseases and cause candidates that cause corresponding diseases are associated in advance. Then, the illness risk calculation unit 127 is only required to calculate, from the second lifestyle pattern data, evaluation values of one or more cause candidates of the specified disease, and calculate a future illness risk using the calculated evaluation values of the one or more cause candidates.

Furthermore, the illness risk calculation unit 127 may calculate an illness risk within one or more future periods. The one or more periods are, for example, periods such as one year, three years, and five years from the present.

Furthermore, the illness risk calculation unit 127 generates an improvement plan of the lifestyle pattern based on the evaluation value of the cause candidate.

The output unit 128 outputs the future illness risk calculated by the illness risk calculation unit 127. For example, by generate presentation data including a future illness risk and transmitting the presentation data to the terminal device 40 using the communication unit 110, the output unit 128 may cause the terminal device 40 to output the presentation data.

The memory 130 includes a nonvolatile storage device such as a flash memory, and stores user data, behavior history data, apparatus data, operation history data, standard lifestyle pattern data, structure data, gene analysis data, and a cause candidate database.

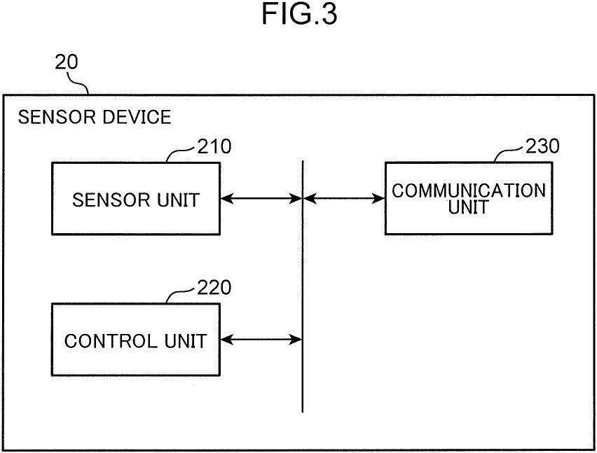

Next, details of the sensor device 20 will be described. FIG. 3 is a block diagram illustrating an example of the configuration of the sensor device 20. The sensor device 20 includes a sensor unit 210, a control unit 220, and a communication unit 230.

The sensor unit 210 includes, for example, a GPS sensor, a biological sensor, and an image sensor, and measures sensing data at a predetermined sampling cycle. The biological sensor measures biological data of the user. The biological data includes a heart rate, an exercise amount, burned calories, consumed calories, presence or absence of smoking, and an alcohol intake amount. Examples of the biological sensor include a heart rate sensor, an acceleration sensor, a gyro sensor, an image sensor, and an odor sensor. The GPS sensor measures position data of the user who owns the sensor device 20. The heart rate sensor measures the heart rate of the user. The acceleration sensor and a three-axis gyro sensor measure the exercise amount and burned calories of the user. The image sensor measures consumed calories and an alcohol intake amount of the user. The odor sensor detects an odor of tobacco.

The control unit 220 includes, for example, a processor such as a CPU, and performs overall control of the sensor device 20. For example, the control unit 220 transmits sensing data measured at a predetermined sampling cycle by the sensor unit 210 to the server 10 using the communication unit 230.

The communication unit 230 includes a communication circuit that connects the sensor device 20 to the network 50. The communication unit 230 transmits the sensing data measured by the sensor unit 210 to the server 10 under the control of the control unit 220.

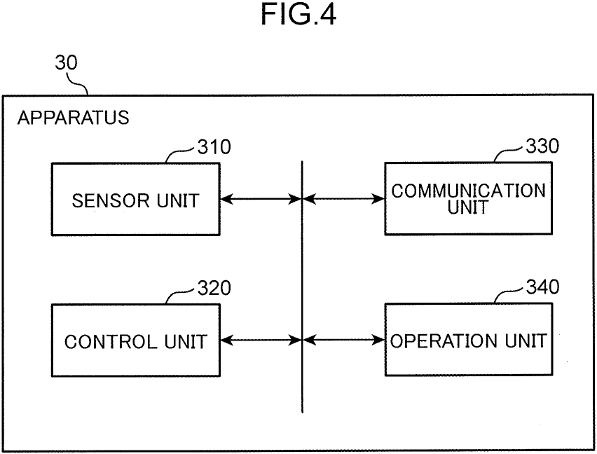

Next, the configuration of the apparatus 30 will be described. FIG. 4 is a block diagram illustrating an example of the configuration of the apparatus 30. The apparatus 30 includes a sensor unit 310, a control unit 320, a communication unit 330, and an operation unit 340.

The sensor unit 310 varies depending on the type of the apparatus 30. For example, if the apparatus 30 is an air conditioning apparatus, the sensor unit 310 includes a temperature sensor that measures the temperature of the surrounding environment and a temperature sensor that measures the temperature of the refrigerant. If the apparatus 30 is a cooking apparatus or a refrigerator, the sensor unit 310 includes a temperature sensor that measures the temperature inside the refrigerator.

The control unit 320 includes a processor such as a CPU and performs overall control of the apparatus 30. For example, the control unit 320 controls the apparatus 30 based on sensing data measured by the sensor unit 310, an operation from the user input by the operation unit 340, and the like. The control unit 320 generates operation data of the apparatus 30 at a predetermined sampling cycle from the state of the apparatus 30 or the like, and transmits the generated operation data to the server 10 using the communication unit 330.

The communication unit 330 is a communication circuit for connecting the apparatus 30 to a network. The communication unit 330 transmits the operation data generated by the control unit 320 to the server 10. The operation unit 340 includes an operation device such as a touchscreen or an input button, for example, and receives an operation from the user. The operation data includes, for example, operation values such as power-on, power-off, and setting content.

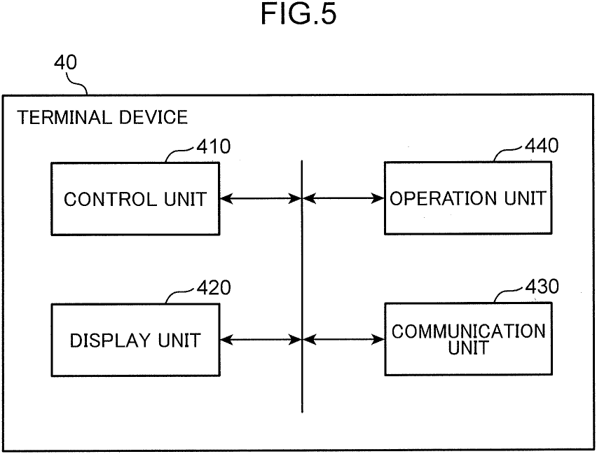

Next, the terminal device 40 will be described. FIG. 5 is a block diagram illustrating an example of the configuration of the terminal device 40. The terminal device 40 includes a control unit 410, a display unit 420, an operation unit 440, and a communication unit 430.

The control unit 410 includes a processor such as a CPU and performs overall control of the terminal device 40. When the communication unit 430 receives the presentation data transmitted from the server 10, the control unit 410 causes the display unit 420 to display the presentation data.

The display unit 420 includes, for example, a display device such as a liquid crystal display panel and an organic EL panel, and displays presentation data under the control of the control unit 410.

The communication unit 430 is a communication circuit for connecting the terminal device 40 to the network 50. The communication unit 430 receives the presentation data transmitted from the server 10.

The operation unit 440 includes operation devices such as a touchscreen, a keyboard, and a mouse, and receives an operation from the user.

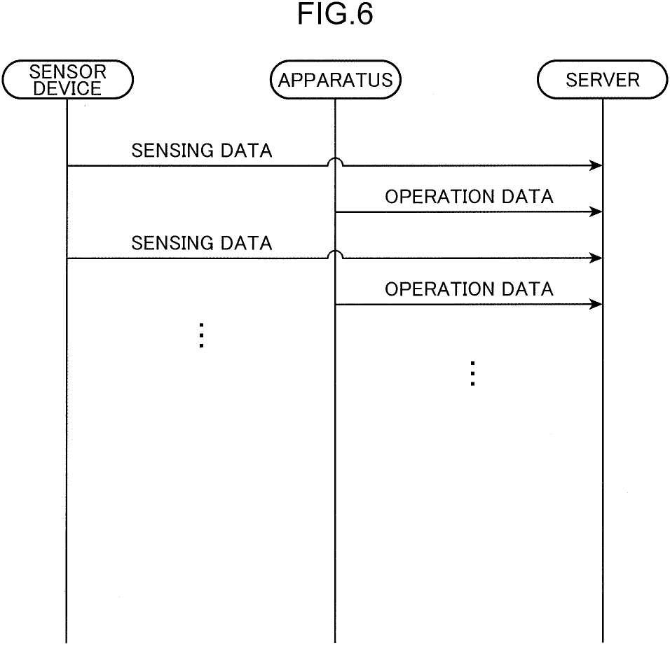

FIG. 6 is a sequence diagram illustrating data transmission and reception in the sensor device 20, the apparatus 30, and the server 10. As illustrated in FIG. 6, the sensor device 20 generates sensing data at a predetermined sampling cycle and transmits the sensing data to the server 10. The apparatus 30 generates operation data at a predetermined sampling cycle and transmits the operation data to the server 10. Here, it has been assumed that the operation data is transmitted at a predetermined sampling cycle, but the present disclosure is not limited to this, and the operation data may be transmitted when a predetermined event occurs. The predetermined event is, for example, power-on or power-off of the apparatus 30, a change in the state of the apparatus 30, or the like. In this manner, the server 10 can accumulate the sensing data into the memory 130 as behavior history data of the user, and can accumulate the operation data into the memory 130 as operation history data of the apparatus 30.

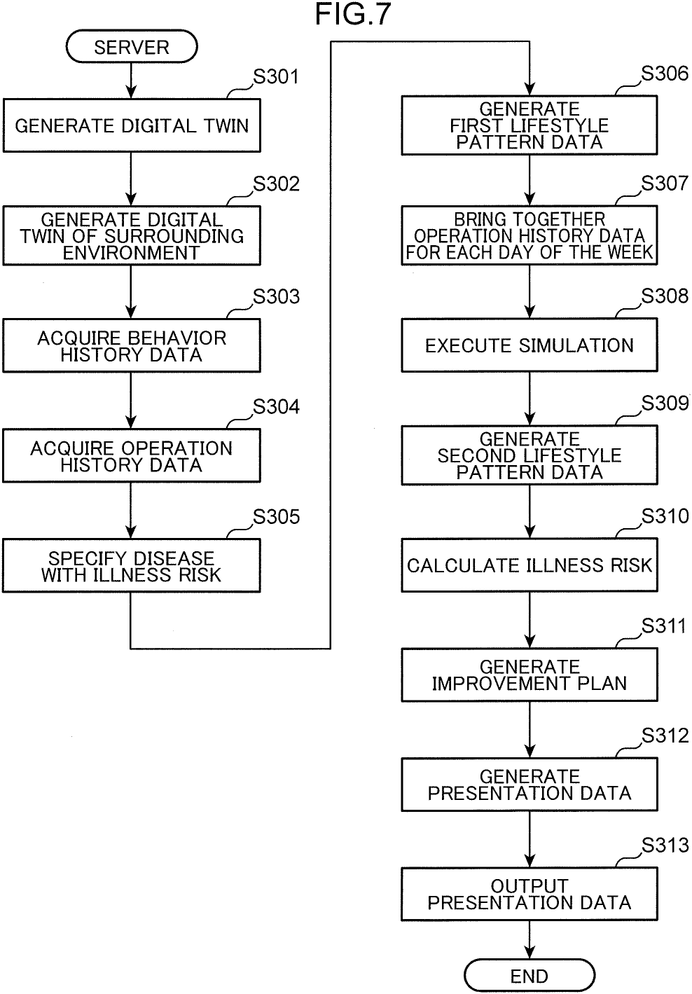

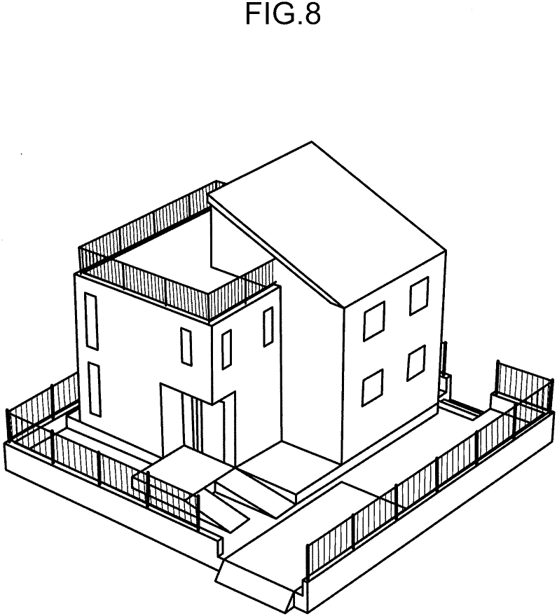

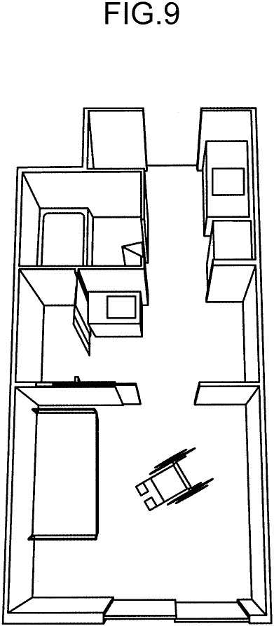

FIG. 7 is a flowchart presenting an example of the processing of the server 10 illustrated in FIG. 1. First, in step S301, the digital twin generation unit 121 generates a digital twin of the user in the cyberspace by using the user data stored in the memory 130, and generates a digital twin of the residence of the user in the cyberspace by using the structure data stored in the memory 130. FIG. 8 is a view illustrating an example of an appearance of the digital twin of the residence. FIG. 9 is a view illustrating an example of a floor plan of the digital twin of the residence. As illustrated in FIG. 8, the digital twin of the residence is three-dimensional modeling data generated using the structure data of the user’s residence. Therefore, windows and doors are arranged in the residential building as per the actual building, and the appearance is reproduced realistically. In the digital twin of the residence, not only the building of the residence but also the site of the residence, a plant planted in the site, a fence surrounding the site, and the like are realistically reproduced.

Furthermore, as illustrated in FIG. 9, the digital twin of the residence also has the three-dimensionally reproduced floor plan in the residence. In the example of FIG. 9, spaces in the residence of a living room, a toilet, a bathroom, a kitchen, and a closet are reproduced, and furniture arranged in the residences is also reproduced.

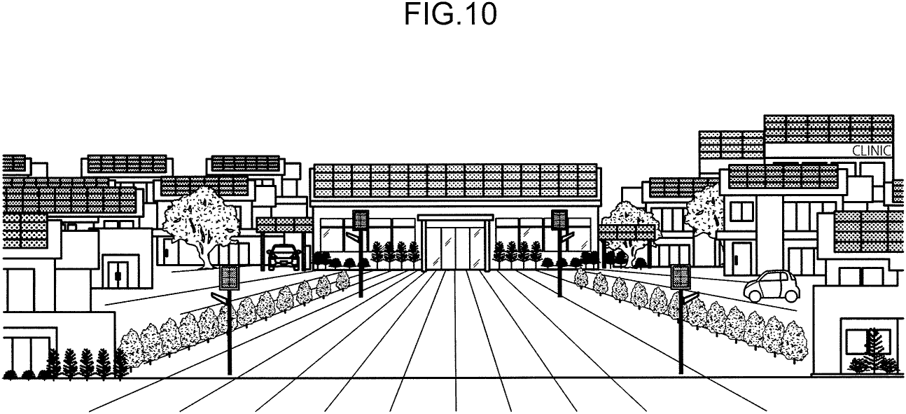

In step S302, the digital twin generation unit 121 generates a digital twin of the region using the structure data of the region including the residence of the user stored in the memory 130. FIG. 10 is a view illustrating an example of a digital twin of the region. This region may be, for example, a zone within a certain range including the residence of the user, or may be a town, a city, or the like to which the residence belongs. As illustrated in FIG. 10, the digital twin of the region includes, in addition to residences in the region, for example, structures existing in the actual region such as a road, a commercial facility, and a street lamp in the region.

In step S303, the acquisition unit 122 acquires, from the memory 130, the behavior history data of a past certain period stored in the memory 130. In the following description, the past certain period is, for example, 3 years, 5 years, 7 years, 10 years, or the like, and is not particularly limited.

In step S304, the acquisition unit 122 acquires, from the memory 130, the operation history data of a past certain period stored in the memory 130. The past certain period of the operation history data is the same period as the past certain period of the behavior history data.

In step S305, the specification unit 123 specifies a disease whose illness risk the user has from the gene analysis data stored in the memory 130. Due to this, it is specified whether or not the user who has provided the gene analysis data is a user who is prone to have each of diseases such as arteriosclerosis, hypertension, diabetes, osteoporosis, and dementia.

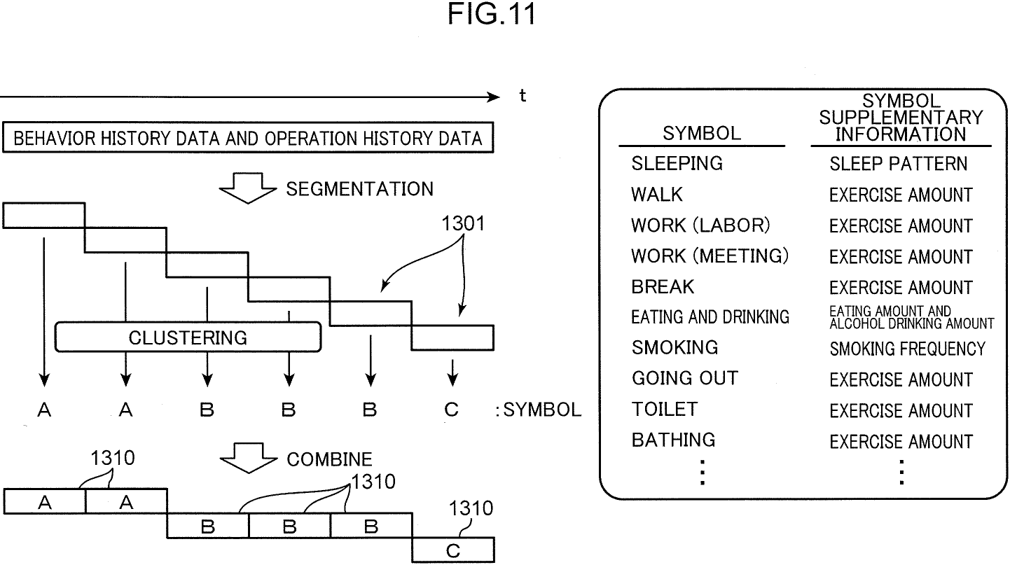

In step S306, the first generation unit 124 analyzes the behavior history data and the operation history data in the past certain period, and generates first lifestyle pattern data indicating the lifestyle pattern of the user up to the present. FIG. 11 is an explanatory view of the generation processing of the first lifestyle pattern data. First, the first generation unit 124 divides each of the behavior history data and the operation history data in the past certain period in time series order for each predetermined period, thereby dividing the behavior history data and the operation history data into an initial segment 1301. The predetermined period is, for example, an appropriate time such as 30 seconds, 1 minute, 10 minutes, or 30 minutes, and is not particularly limited. Next, the first generation unit 124 performs clustering on each initial segment 1301 and gives each initial segment 1301 a symbol indicating the user’s behavior.

For example, the first generation unit 124 inputs the behavior history data and the operation history data included in each initial segment 1301 to a machine learning model having the behavior history data and the operation history data as explanatory variables and the behavior of the user as an objective variable, the machine learning model obtained by performing machine learning in advance, and specifies the behavior of the user. Then, the first generation unit 124 is only required to give each initial segment 1301 a symbol indicating the specified behavior. Alternatively, the first generation unit 124 may perform clustering using a technique such as a k-means method or a random forest, and give a symbol to each initial segment 1301 based on a clustering result. A list of symbols given to each initial segment 1301 is illustrated on the right side of FIG. 11.

The symbol is data indicating behavior of the user, and is, for example, sleeping, walk, work (labor), work (meeting), break, eating and drinking, smoking, going out, toilet, bathing, and the like. In addition to the symbol, symbol supplementary information is also given to each initial segment 1301. The symbol supplementary information for sleep indicates sleep patterns such as REM sleep and non-REM sleep. The information indicating the sleep pattern is specified from, for example, a sensor value (for example, exercise amount, heart rate, and the like) included in the initial segment 1301 to which the sleep symbol is given. The symbol supplementary information for each of walk, work (labor), work (meeting), break, going out, toilet, and bathing is, for example, an exercise amount. The exercise amount is specified, for example, from a sensor value (for example, an angular velocity measured by a gyro sensor or an acceleration measured by an acceleration sensor) included in the initial segment 1301 to which a symbol related to the exercise amount is given. Note that, as the exercise amount, the angular velocity of the user or the acceleration of the user may be adopted, a value obtained by multiplying the weight of the user by the speed may be adopted, or burned calories may be adopted.

The symbol supplementary information for eating and drinking is an eating amount and an alcohol drinking amount. The eating amount is specified from a sensor value (for example, consumed calories) included in the initial segment 1301 to which the eating and drinking symbol is given. The consumed calories can be obtained, for example, by analyzing the dish eaten by the user from image data of the user while eating. The alcohol drinking amount is specified from, for example, a sensor value (for example, alcohol intake amount) included in the initial segment 1301 to which the eating and drinking symbol is given.

The symbol supplementary information of smoking is a smoking frequency. The presence or absence of smoking is detected by associating the detection result of the tobacco odor by the odor sensor with the position data of the user. The smoking frequency is, for example, the number of cigarettes smoked.

Next, the first generation unit 124 combines one or more initial segments 1301 that are the initial segments 1301 to which the same symbol is given and are continuous in time series. In this example, two initial segments 1301 to which a symbol “A” is given are combined to generate a segment 1310, three initial segments 1301 to which a symbol “B” is given are combined to generate a segment 1310, and a segment 1310 including one initial segment 1301 to which a symbol “C” is given is generated.

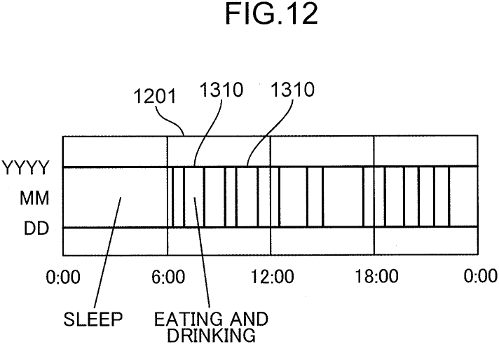

FIG. 12 is a view illustrating an example of the data configuration of lifestyle pattern data 1201 of each day. The daily lifestyle pattern data 1201 includes one or more segments 1310 arranged in time series in time slots of 24 hours from 0:00 to 24:00 (0:00). In this example, the segment 1310 having the sleep symbol is arranged in the time slot from 0:00 to 6:30, and the segment 1310 having the eating and drinking symbol is arranged in the time slot from 6:30 to 7:30.

The lifestyle pattern data 1201 of each day is associated with date data including year (YYYY), month (MM), and day (DD). In this manner, it is understood that the lifestyle pattern data 1201 of each day is data expressing the behavior of the user of each day in a past certain period in time series.

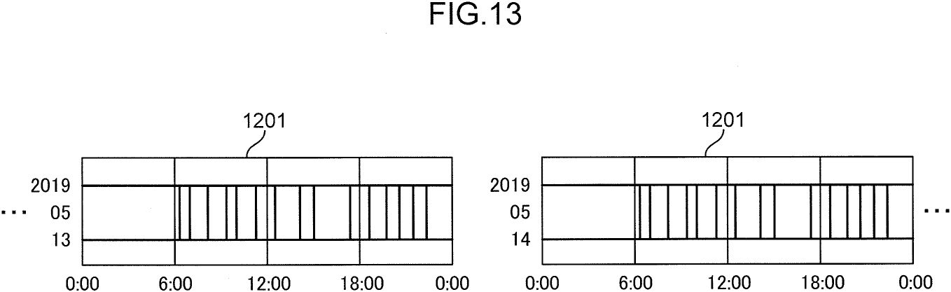

FIG. 13 is a view illustrating an example of the data configuration of lifestyle pattern data generated by the first generation unit 124. As illustrated in FIG. 13, the first generation unit 124 generates the daily lifestyle pattern data 1201 for a past certain period such as the lifestyle pattern data 1201 for May 13, 2019 and the lifestyle pattern data 1201 for May 14, 2019.

Next, the first generation unit 124 brings together the lifestyle pattern data 1201 of the past certain period for each day of the week to generate lifestyle pattern data for each day of the week. In this case, the first generation unit 124 divides the time slot from, for example, 0:00 to 24:00 into the initial segment 1301, votes symbols constituting the lifestyle pattern data classified for each day of the week for the initial segment 1301 to which the time slot corresponds, and determines representative behavior in each initial segment 1301 from the voting result. Then, the first generation unit 124 generates lifestyle pattern data for each day of the week by combining one or more initial segments 1301 having the same symbol and continuous in time series and generating the segment 1310. Thus, the first lifestyle pattern data including the lifestyle pattern data for each day of the week is generated.

Refer back to FIG. 7. In step S307, the first generation unit 124 brings together the operation history data for each day of the week. For example, as illustrated in FIG. 11, the first generation unit 124 is only required to divide the operation history data into a plurality of initial segments 1301, and is only required to obtain an average value for each type of sensor value in each initial segment 1301 to generate the operation history data for each day of the week.

In step S308, using the first lifestyle pattern data and the standard lifestyle pattern data, the simulation execution unit 125 executes the above-described simulation of causing the digital twin of the user and the digital twin of the apparatus 30 to operate in the cyberspace.

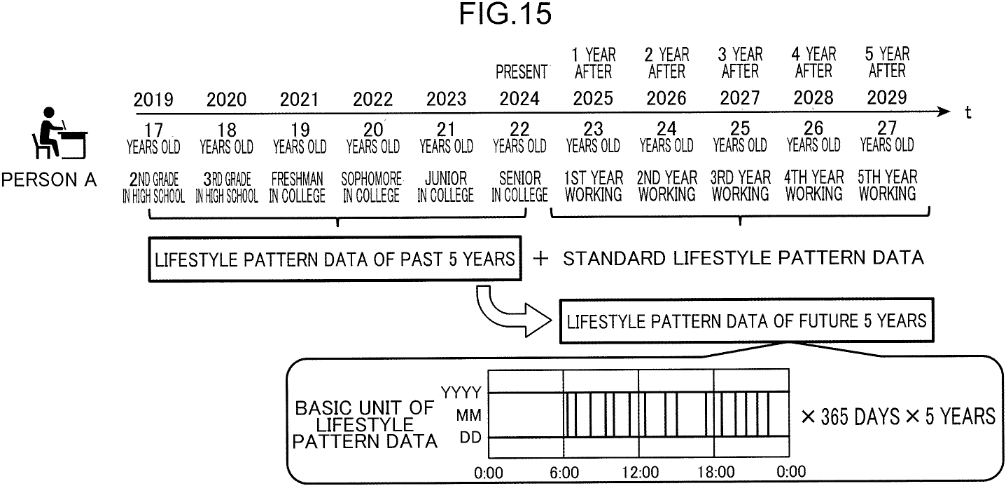

FIG. 15 is an explanatory view of simulation. FIG. 15 illustrates an example of a simulation of a person A, who is 22 years old and a college senior at the present in 2024. This simulation uses the first lifestyle pattern data for the past 5 years from 2019 to 2023 of the person A. This first lifestyle pattern data is corrected in accordance with the standard lifestyle pattern data, and the digital twin of the person A is operated in the cyberspace in accordance with the corrected first lifestyle pattern data. At this time, the apparatus 30 is also operated in accordance with the apparatus history data for the past 5 years. Here, the simulation is executed in the future 5 years from 2025 to 2029, that is, in the period from the first year working to the fifth year working.

For example, in a case where the person A executes a simulation of one day when the person A is 25 years old, the simulation execution unit 125 corrects the first lifestyle pattern data of the day of the week (for example, Tuesday) of the corresponding day by using the standard lifestyle pattern data of Tuesday when the person A is 25 years old. Then, the simulation execution unit 125 causes the digital twin of the user to operate in the cyberspace using the corrected first lifestyle pattern data.

For example, when the sleep hours indicated by the standard lifestyle pattern data of 25 years old is lower by x% than the sleep hours indicated by the first lifestyle pattern data, the simulation execution unit 125 corrects the first lifestyle pattern data so that the sleep hours becomes shorter by x%.

For example, when the standard lifestyle pattern data describes that the basal metabolic rate of 25 years old is lowered by y% as compared with the basal metabolic rate of 22 years old, the simulation execution unit 125 corrects the first lifestyle pattern data so that the burned calories or the exercise amount of each behavior becomes lower by y%.

For example, when it is indicates that the daily consumed calories of 25 years old is lowered by z% as compared with the daily consumed calories of 25 years old, the simulation execution unit 125 corrects the first lifestyle pattern data so that the consumed calories indicated by the standard lifestyle pattern data of 25 years old is lowered by z%.

Furthermore, by using the operation history data brought together for each day of the week, the simulation execution unit 125 causes the digital twin of the apparatus 30 to operate. For example, in a case of executing a simulation for a certain day, the simulation execution unit 125 causes the digital twin of the apparatus 30 to operate in the cyberspace by using the operation history data corresponding to the day of the week of the day.

Furthermore, the simulation execution unit 125 monitors the behavior content of the digital twin of the user and the operation content of the digital twin of the apparatus 30, and records, in time series at a predetermined sampling cycle, the behavior history data indicating the monitored behavior content and the operation history data indicating the operation content, thereby generating a simulation execution result. The behavior content to be monitored includes, for example, position data of the digital twin of the user, and biological data of the user. The operation content to be monitored includes, for example, an operation value of the digital twin of the apparatus 30.

Refer back to FIG. 7. In step S309, the second generation unit 126 generates the second lifestyle pattern data from the simulation execution result. In the example of FIG. 15, lifestyle pattern data for each day for 5 years in the future is generated.

Similarly to the first generation unit 124, the second generation unit 126 is only required to generate the second lifestyle pattern data using the technique illustrated in FIG. 11. That is, the second generation unit 126 divides the behavior history data and the operation history data included in the simulation execution result into the initial segments 1301, and performs clustering on each initial segment 1301, thereby giving a symbol to each initial segment 1301. Here, the symbol given is the same as that in the first lifestyle pattern data. Then, the second generation unit 126 combines the initial segments 1301 to which the same symbol is given. The second generation unit 126 performs this processing on the behavior history data and the operation history data of each day in the future, thereby generating lifestyle pattern data of each day for 5 years in the future.

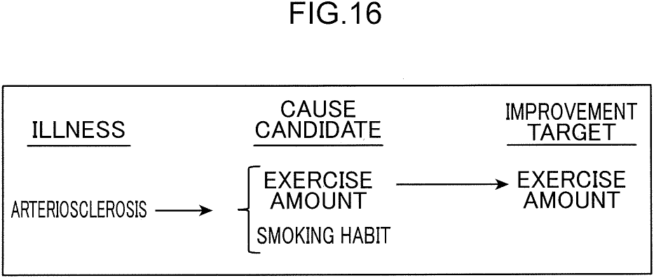

Refer back to FIG. 7. In step S310, the illness risk calculation unit 127 calculates a future illness risk for the disease specified in step S305 based on the second lifestyle pattern data. FIG. 16 is an explanatory view of the processing for calculating the future illness risk. Hereinafter, the description assumes that the disease specified in step S305 is arteriosclerosis. The illness risk calculation unit 127 refers to the cause candidate database stored in the memory 130 and specifies a cause candidate associated with arteriosclerosis.

Here, the cause candidates of arteriosclerosis are exercise amount and smoking habit. In this case, the illness risk calculation unit 127 is only required to calculate, from the second lifestyle pattern data, an exercise evaluation value, which is an evaluation value regarding exercise of the digital twin of the user, and a smoking evaluation value regarding the smoking habit, calculate a comprehensive evaluation value from the both evaluation values, and calculate the comprehensive evaluation value as a future illness risk. Here, the exercise evaluation value assumes a value from 0 to 1, for example, and increases with an increase in average daily burned calories or exercise amount of the digital twin of the user. The smoking evaluation value assumes a value of, for example, equal to or more than 0 to equal to or less than 1, and increases with a decrease in average daily value of the number of cigarettes smoked of the digital twin of the user. The comprehensive evaluation value is an average value of, for example, the exercise evaluation value and the smoking evaluation value.

Furthermore, the illness risk calculation unit 127 calculates an illness risk within one or more future periods based on the comprehensive evaluation value. For example, the illness risk calculation unit 127 is only required to calculate the illness risk within one or more future periods by correcting the comprehensive evaluation value by using a predetermined arithmetic expression that increases the comprehensive evaluation value as time elapses.

Here, while arteriosclerosis has been exemplified, the illness risk is similarly calculated for other diseases (hypertension, diabetes, osteoporosis, dementia, and the like). That is, the illness risk calculation unit 127 specifies a cause candidate corresponding to the disease with reference to the cause candidate database, calculates an evaluation value for each specified cause candidate, and calculates a comprehensive evaluation value from each evaluation value.

Refer back to FIG. 7. In step S311, the illness risk calculation unit 127 generates an improvement plan based on the evaluation value for each cause candidate. Refer to FIG. 16. In the case of FIG. 16, the smoking evaluation value is higher than a threshold value, but the exercise evaluation value is lower than the threshold value, and therefore the exercise amount is specified as an improvement target. In this manner, the illness risk calculation unit 127 is only required to compare the evaluation value for each cause candidate with the threshold value, and specify, as an improvement target of lifestyle pattern, a cause candidate having an evaluation value for each cause candidate lower than the threshold value. As the threshold value, for example, an evaluation value for each cause candidate of general people of the same generation is adopted.

Refer back to FIG. 7. In step S312, the illness risk calculation unit 127 generates presentation data including an illness risk and an improvement plan within one or more future periods.

In step S313, the output unit 128 outputs the presentation data. Here, the output unit 128 is only required to transmit the presentation data to the terminal device 40 using the communication unit 110. The terminal device 40 that has received the presentation data displays the presentation data on the display unit 420.

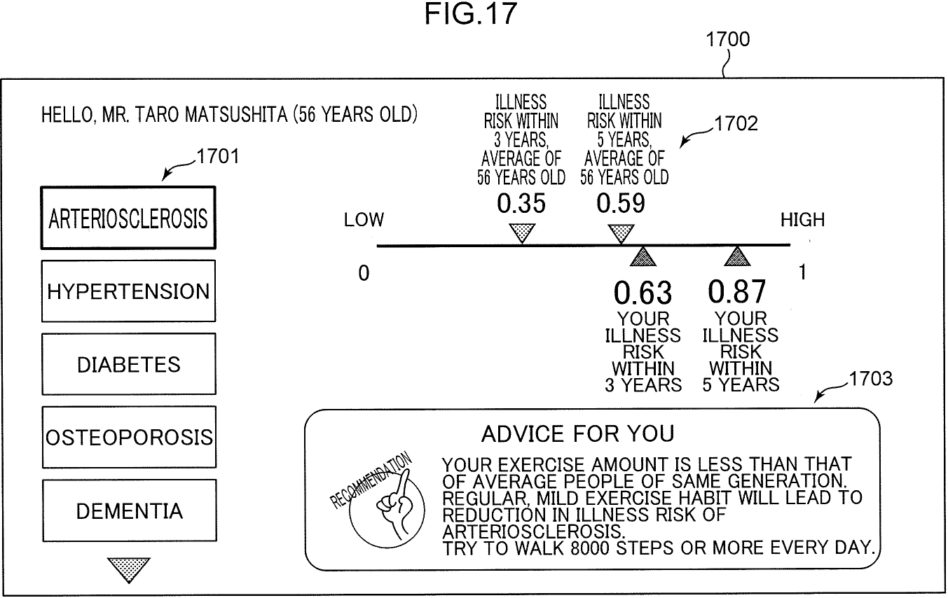

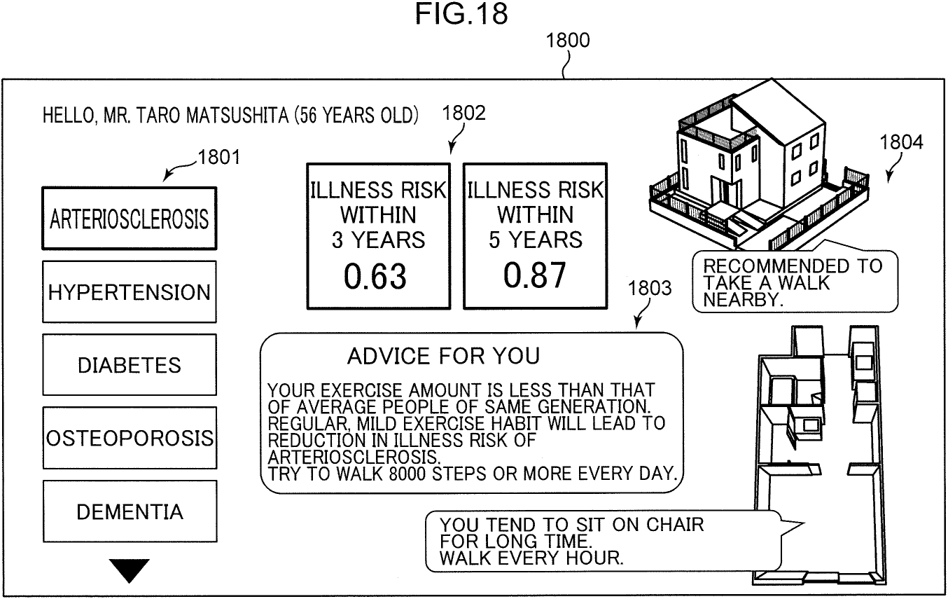

FIG. 17 is a view illustrating a presentation screen 1700. The presentation screen 1700 is a display screen of the presentation data. The same applies to the presentation screens of FIGS. 18 to 20. Here, the presentation screen 1700 of Mr. Taro Matsushita who is 56 years old is displayed. The presentation screen 1700 includes a disease display field 1701, an illness risk display field 1702, and an improvement plan display field 1703.

The disease display field 1701 displays diseases that the user has been determined to possibly have among a plurality of diseases. Here, the outer frame of arteriosclerosis is displayed thicker than the outer frames of the other diseases because among arteriosclerosis, hypertension, diabetes, osteoporosis, and dementia, arteriosclerosis has been specified as a disease that the user will possibly has.

The illness risk display field 1702 displays a future illness risk. Here, the illness risk in each period of within 3 years and within 5 years is displayed for each of the corresponding user and general people. In this example, the illness risk of the user within 3 years is displayed as 0.63, and the illness risk within 5 years is displayed as 0.87. On the other hand, the illness risks within 3 years and within 5 years of general people at the age of 56 are displayed as 0.35 and 0.59, respectively. Therefore, this user can recognize that the risk of having arteriosclerosis is higher than that of general people.

The improvement plan display field 1703 displays an improvement plan of the lifestyle pattern. This user has an exercise evaluation value lower than an evaluation value (threshold value) of general people of the same generation. Therefore, advice for encouraging an exercise habit is displayed in the improvement plan display field 1703.

FIG. 18 is a view illustrating a presentation screen 1800 according to another example. The presentation screen 1800 includes a disease display field 1801, an illness risk display field 1802, an improvement plan display field 1803, and a detail display field 1804.

The disease display field 1801 and the improvement plan display field 1803 are the same as the disease display field 1701 and the improvement plan display field 1703. Although the illness risk display field 1702 also displays the future illness risk of general people of the same generation as the user, the illness risk display field 1802 only displays the future illness risk of the user. Here, the illness risks of the user within 3 years and within 5 years are each displayed. The detail display field 1804 displays a supplementary explanation of the improvement plan described in the improvement plan display field 1803. Here, an external view of the residence is displayed, and advice for recommending to take a walk around the residence is displayed in the detail display field 1804. Furthermore, the floor plan of the residence is displayed, and since the time during which the use is sitting on a chair is long, advice for recommending to take a walk every 1 hour is displayed in the detail display field 1804. The external view of the residence and the floor plan of the residence displayed in the detail display field 1804 are generated based on the digital twin of the user’s residence generated by the digital twin generation unit 121.

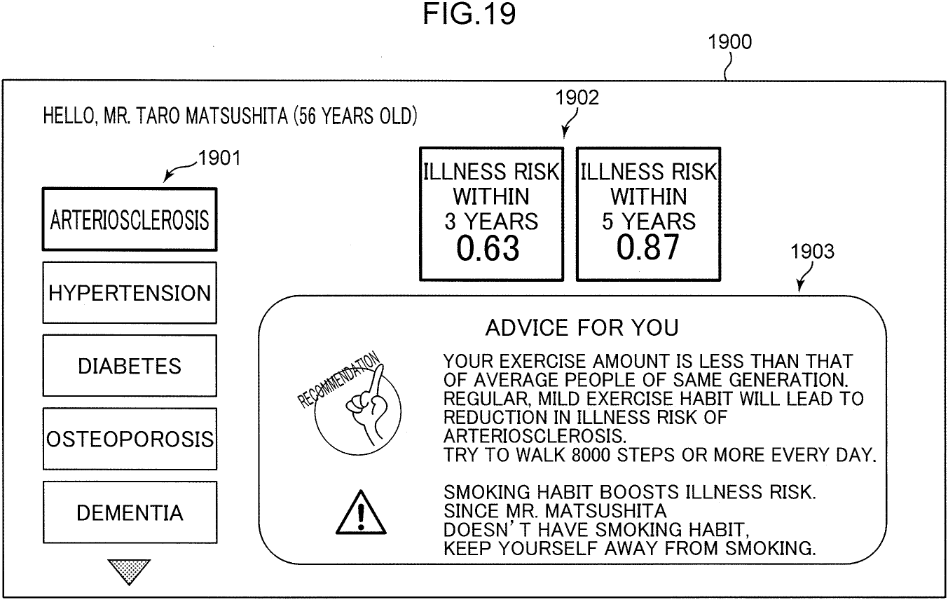

FIG. 19 is a view illustrating a presentation screen 1900 according to still another example. The presentation screen 1900 includes a disease display field 1901, an illness risk display field 1902, and an improvement plan display field 1903. The disease display field 1901 and the illness risk display field 1902 are the same as the disease display field 1801 and the illness risk display field 1802.

The improvement plan display field 1903 further displays cautions on the lifestyle in addition to advice for recommending an exercise habit. Here, since the smoking habit increases the illness risk of arteriosclerosis, advice for refraining from smoking is displayed in the improvement plan display field 1903. Since this user does not have a smoking habit, words in consideration of that are also included in this advice.

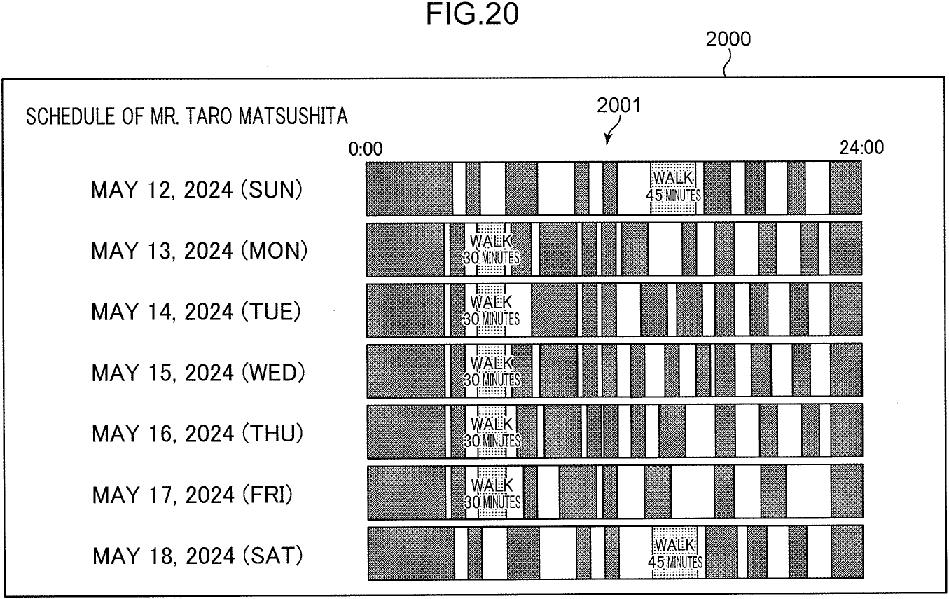

FIG. 20 is a view illustrating a presentation screen 2000 according to yet another example. The presentation screen 2000 includes a schedule display field 2001. The schedule display field 2001 displays, in units of one day, the schedule of the user for the last one week. This schedule may be generated, for example, based on the second lifestyle pattern data, or a schedule generated by external schedule software may be used.

Here, the illness risk calculation unit 127 determines that the exercise amount of the user is lower than that of general people of the same generation. Therefore, a walk is included in the schedule in order to encourage an exercise habit. For example, the illness risk calculation unit 127 acquires a schedule of the user for the last one week, and detects an empty time of a predetermined time or more from the acquired schedule of the user. Then, the schedule display field 2001 is generated by including the schedule of a walk into the detected empty time. In this example, a 30-minute or 45-minute walk time is scheduled for each day of a week from May 12 (Sunday), 2024 to May 18 (Saturday), 2024.

Due to this, the user can easily improve the lifestyle pattern by taking a walk in accordance with the schedule display field 2001.

In this manner, according to the information processing system 1 according to the present embodiment, a disease that the user is likely to have is specified from the gene analysis data of the user. The first lifestyle pattern data indicating the lifestyle pattern of the user up to the present is generated from the behavior history data of the user and the operation history data of the apparatus. A simulation of causing the digital twin of the user and the digital twin of the apparatus to operate in the cyberspace based on the generated first lifestyle pattern data, the standard lifestyle pattern data in accordance with the future life stage, and the operation history data is executed. The second lifestyle pattern data for predicting the future lifestyle pattern of the user is generated from the simulation execution result. An illness risk of having the specified disease in the future is calculated based on the generated second lifestyle pattern data, and the calculated illness risk is output. Therefore, the present configuration can predict the illness risk of the disease that the user is likely to have in the future. By presenting the future illness risk to the user, it is possible to give the user an opportunity to review the current lifestyle pattern. This allows the user to reduce the future illness risk.

The following modifications can be adopted in the present disclosure.

(1) In the flowchart of FIG. 7, step S305 for specifying a disease with an illness risk is provided after steps S301 and S302 for generating the digital twin, but its order is optional as long as it is before the simulation is executed. For example, step S305 may be provided before steps S301 and S302.

(2) In the lifestyle pattern data illustrated in FIG. 12 and the like, it has been described that one symbol is allocated to the segment 1310, but the present disclosure is not limited to this, and a plurality of symbols may be allocated. Note that, in a case where a plurality of symbols are allocated, in generating the lifestyle pattern data for each day of the week, the first generation unit 124 is only required to cast a vote using the plurality of symbols as one set and specify representative behavior.

### INDUSTRIAL APPLICABILITY

According to the present disclosure, since the future illness risk of the user is calculated, it is useful in the healthcare industry.

1. An information processing method comprising:
by a computer,
generating, in a cyberspace, a digital twin of a user and a digital twin of an apparatus installed in a residence of the user, based on real-world data;
acquiring behavior history data indicating a behavior history of the user and operation history data indicating an operation history of the apparatus;
specifying a disease that the user is likely to suffer from, based on gene analysis data of the user;
analyzing the behavior history data and the operation history data to generate first lifestyle pattern data indicating a lifestyle pattern of the user up to present;
executing a simulation for causing the digital twin of the user and the digital twin of the apparatus to operate in the cyberspace based on the first lifestyle pattern data, standard lifestyle pattern data indicating a standard lifestyle pattern in accordance with a future life stage, and the operation history data;
generating second lifestyle pattern data in which a future lifestyle pattern of the user is predicted from an execution result of the simulation;
calculating a future illness risk of the user for the specified disease based on the second lifestyle pattern data; and
outputting the illness risk.

2. The information processing method according to claim 1, wherein a digital twin of the residence is included in the cyberspace.

3. The information processing method according to claim 1, further comprising generating an improvement plan of the lifestyle pattern of the user based on the second lifestyle pattern data and the illness risk,
wherein in the outputting, the improvement plan is further output.

4. The information processing method according to claim 3, wherein the improvement plan includes exercise information indicating exercise recommended for reducing the illness risk.

5. The information processing method according to claim 1, wherein in calculating the illness risk, an illness risk within one or more future periods is calculated.

6. The information processing method according to claim 1, wherein the disease is a lifestyle disease.

7. The information processing method according to claim 1, wherein in generating the second lifestyle pattern data, a daily lifestyle pattern from present to a predetermined future time point is predicted.

8. The information processing method according to claim 1, wherein the real-world data includes attribute data of the user and position data of the apparatus.

9. The information processing method according to claim 1, wherein in executing the simulation, a simulation of causing the digital twin of the user to operate in the cyberspace based on the first lifestyle pattern data and the standard lifestyle pattern data and causing the digital twin of the apparatus to operate in the cyberspace based on the operation history data is executed.

10. An information processing device comprising:
a digital twin generation unit that generates, in a cyberspace, a digital twin of a user and a digital twin of an apparatus installed in a residence of the user, based on real-world data;
an acquisition unit that acquires behavior history data indicating a behavior history of the user and operation history data indicating an operation history of the apparatus;
a specification unit that specifies a disease that the user is likely to suffer from, based on gene analysis data of the user;
a first generation unit that analyzes the behavior history data and the operation history data to generate first lifestyle pattern data indicating a lifestyle pattern of the user up to present;
a simulation execution unit that executes a simulation for causing the digital twin of the user and the digital twin of the apparatus to operate in the cyberspace based on the first lifestyle pattern data, standard lifestyle pattern data indicating a standard lifestyle pattern in accordance with a future life stage, and the operation history data;
a second generation unit that generates second lifestyle pattern data in which a future lifestyle pattern of the user is predicted from an execution result of the simulation;
an illness risk calculation unit that calculates a future illness risk of the user for the specified disease based on the second lifestyle pattern data; and
an output unit that outputs the illness risk.

11. A non-transitory computer readable recording medium storing a program that causes a computer to:
generate, in a cyberspace, a digital twin of a user and a digital twin of an apparatus installed in a residence of the user, based on real-world data;
acquire behavior history data indicating a behavior history of the user and operation history data indicating an operation history of the apparatus;
specify a disease that the user is likely to suffer from, based on gene analysis data of the user;
analyze the behavior history data and the operation history data to generate first lifestyle pattern data indicating a lifestyle pattern of the user up to present;
execute a simulation for causing the digital twin of the user and the digital twin of the apparatus to operate in the cyberspace based on the first lifestyle pattern data, standard lifestyle pattern data indicating a standard lifestyle pattern in accordance with a future life stage, and the operation history data;
generate second lifestyle pattern data in which a future lifestyle pattern of the user is predicted, from an execution result of the simulation;
calculate a future illness risk of the user for the specified disease based on the second lifestyle pattern data; and
output the illness risk.
